(19) United States
(12) Patent Application Publication
(10) Pub. No.: US 2024/0127439 A1
(43) Pub. Date: Apr. 18, 2024

(54) APPARATUSES, METHODS, AND SYSTEMS FOR NON-INVASIVE BREATH ANALYSIS

(71) Applicant: HONEYWELL INTERNATIONAL INC., Charlotte, NC (US)

(72) Inventor: Ravi AITAL, Charlotte, NC (US)

(21) Appl. No.: 18/476,437

(22) Filed: Sep. 28, 2023

(30) Foreign Application Priority Data
Oct. 12, 2022 (IN) 202211058063

(51) Int. Cl. G06T 7/00; G16H 50/20

(52) U.S. Cl. CPC G06T 7/0012; G16H 50/20; G06T 2207/10004; G06T 2207/20081; G06T 2207/30004

(57) ABSTRACT

Example apparatuses, methods, and systems for non-invasive breath analysis are provided. An example non-invasive breath analyzer apparatus includes an image generating device and a spectrometric data analyzing device. In some examples, the image generating device is positioned within a breath analyzer housing of the non-invasive breath analyzer and generates exhaled breath digital image data objects. In some examples, the spectrometric data analyzing device is in electronic communication with the image generating device and includes a processor and a memory storing non-transitory program code.

### CROSS-REFERENCE TO RELATED DOCUMENTS

This application claims priority pursuant to 35 U.S.C. 119(a) to Indian Application No. 202211058063, filed Oct. 12, 2022, which application is incorporated herein by reference in its entirety.

### FIELD OF THE INVENTION

The present disclosure relates generally to apparatuses, systems, and methods for non-invasive analysis of breath based at least in part on laser spectroscopy.

For example, various embodiments of the present disclosure provide an example non-invasive breath analyzer apparatus that comprises an image generating device and a spectrometric data analyzing device. In some embodiments, the spectrometric data analyzing device generates spectrometric-based prediction data objects based at least in part on exhaled breath digital image data objects from the image generating device.

### BACKGROUND

Applicant has identified many deficiencies and problems associated with many methods, apparatus, and systems related to analyzing breath from users. For example, many methods, apparatus, and systems are not capable of providing accurate analysis of exhaled breath from users.

### BRIEF SUMMARY

Various embodiments described herein relate to methods, apparatuses, and systems that provide technical advantages and benefits on non-invasive breath analysis.

In accordance with various embodiments of the present disclosure, a non-invasive breath analyzer apparatus is provided. In some embodiments, the non-invasive breath analyzer apparatus comprises an image generating device and a spectrometric data analyzing device.

In some embodiments, the image generating device is positioned within a breath analyzer housing and generates an exhaled breath digital image data object.

In some embodiments, the spectrometric data analyzing device is in electronic communication with the image generating device and comprises a processor and a memory storing non-transitory program code. In some embodiments, the memory and the non-transitory program code are configured to, with the processor, cause the spectrometric data analyzing device to receive the exhaled breath digital image data object from the image generating device, generate a plurality of exhaled breath spectrometric data objects based at least in part on the exhaled breath digital image data object, input the plurality of exhaled breath spectrometric data objects to at least one trained machine learning computing model, receive at least one spectrometric-based prediction data object from the at least one trained machine learning computing model, and perform one or more prediction-based data operations based at least in part on the at least one spectrometric-based prediction data object.

In some embodiments, when generating the plurality of exhaled breath spectrometric data objects, the memory and the non-transitory program code are configured to, with the processor, cause the spectrometric data analyzing device to extract at least one of photographic metadata or spectrometric metadata from the exhaled breath digital image data object; and generate the plurality of exhaled breath spectrometric data objects based at least in part on the at least one of photographic metadata or spectrometric metadata.

In some embodiments, the at least one trained machine learning computing model comprises at least one trained classification-based estimation model.

In some embodiments, prior to inputting the plurality of exhaled breath spectrometric data objects to the at least one trained machine learning computing model, the memory and the non-transitory program code are configured to, with the processor, cause the spectrometric data analyzing device to train at least one classification-based estimation model.

In some embodiments, when training the at least one classification-based estimation model, the memory and the non-transitory program code are configured to, with the processor, cause the spectrometric data analyzing device to: retrieve a plurality of training exhaled breath spectrometric data objects associated with a training spectrometric-based prediction data object; input the plurality of training exhaled breath spectrometric data objects to the at least one classification-based estimation model; receive a testing spectrometric-based prediction data object from the at least one classification-based estimation model; and adjust the at least one classification-based estimation model based at least in part on the testing spectrometric-based prediction data object and the training spectrometric-based prediction data object.

In some embodiments, when performing the one or more prediction-based data operations based at least in part on the at least one spectrometric-based prediction data object, the memory and the non-transitory program code are configured to, with the processor, cause the spectrometric data analyzing device to: determine whether the at least one spectrometric-based prediction data object satisfies a health condition threshold; and in response to determining that the at least one spectrometric-based prediction data object satisfies the health condition threshold, transmit a predicted health condition indication to a non-invasive breath analyzer server.

In some embodiments, when performing the one or more prediction-based data operations based at least in part on the at least one spectrometric-based prediction data object, the memory and the non-transitory program code are configured to, with the processor, cause the spectrometric data analyzing device to: retrieve a previous spectrometric-based prediction data object associated with a previous time point; retrieve a subsequent spectrometric-based prediction data object associated with a subsequent time point; and generate a predicted condition progression indication based at least in part on comparing the subsequent spectrometric-based prediction data object with the previous spectrometric-based prediction data object.

In accordance with various embodiments of the present disclosure, a computer program product for non-invasive breath analysis is provided. In some embodiments, the computer program product comprises at least one non-transitory computer-readable storage medium having computer-readable program code portions stored therein. In some embodiments, the computer-readable program code portions comprise an executable portion configured to: receive an exhaled breath digital image data object from an image generating device; generate a plurality of exhaled breath spectrometric data objects based at least in part on the exhaled breath digital image data object; input the plurality of exhaled breath spectrometric data objects to at least one trained machine learning computing model; receive at least one spectrometric-based prediction data object from the at least one trained machine learning computing model; and perform one or more prediction-based data operations based at least in part on the at least one spectrometric-based prediction data object.

In accordance with various embodiments of the present disclosure, a computer-implemented method is provided. In some embodiments, the computer-implemented method comprises receiving an exhaled breath digital image data object from an image generating device; generating a plurality of exhaled breath spectrometric data objects based at least in part on the exhaled breath digital image data object; inputting the plurality of exhaled breath spectrometric data objects to at least one trained machine learning computing model; receiving at least one spectrometric-based prediction data object from the at least one trained machine learning computing model; and performing one or more prediction-based data operations based at least in part on the at least one spectrometric-based prediction data object.

The foregoing illustrative summary, as well as other exemplary objectives and/or advantages of the disclosure, and the manner in which the same are accomplished, are further explained in the following detailed description and its accompanying drawings.

### BRIEF DESCRIPTION OF THE DRAWINGS

The description of the illustrative embodiments may be read in conjunction with the accompanying figures. It will be appreciated that, for simplicity and clarity of illustration, elements illustrated in the figures have not necessarily been drawn to scale, unless described otherwise. For example, the dimensions of some of the elements may be exaggerated relative to other elements, unless described otherwise. Embodiments incorporating teachings of the present disclosure are shown and described with respect to the figures presented herein, in which:

FIG. 1 is an example schematic diagram illustrating an example non-invasive breath analysis platform in accordance with some embodiments of the present disclosure;

FIG. 2 is an example schematic diagram illustrating at least an example portion of an example non-invasive breath analyzer apparatus in accordance with some embodiments of the present disclosure;

FIG. 3 is an example block diagram illustrating at least some example components of an example non-invasive breath analyzer apparatus in accordance with some embodiments of the present disclosure;

FIG. 4 is an example block diagram illustrating at least some example components of an example non-invasive breath analyzer server in accordance with some embodiments of the present disclosure;

FIG. 5 is an example flow diagram illustrating an example method in accordance with some embodiments of the present disclosure;

FIG. 6 is an example flow diagram illustrating an example method in accordance with some embodiments of the present disclosure;

FIG. 7 is an example flow diagram illustrating an example method in accordance with some embodiments of the present disclosure;

FIG. 8 is an example flow diagram illustrating an example method in accordance with some embodiments of the present disclosure; and

FIG. 9 is an example flow diagram illustrating an example method in accordance with some embodiments of the present disclosure.

### DETAILED DESCRIPTION OF THE INVENTION

Some embodiments of the present disclosure will now be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements Like numbers refer to like elements throughout.

The phrases “in one embodiment,” “according to one embodiment,” “in some embodiments,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

The term “electronically coupled,” “electronically coupling,” “electronically couple,” “in communication with,” “in data communication with,” “in electronic communication with,” or “connected” in the present disclosure refers to two or more elements or components being connected through wired means and/or wireless means, such that signals, voltage/current, data and/or information may be transmitted to and/or received from these elements or components.

As described above, there are many technical challenges and difficulties associated with analyzing exhaled breath from users. For example, many methods, apparatus, and systems are not capable of analyzing exhaled breath from users. Some methods may analyze exhaled breath from users by passing exhaled breath through a chemical solution or reagent. However, such methods do not capture all available data and/or information from the exhaled breath, and therefore cannot provide accurate analysis of the exhaled breath.

Various embodiments of the present disclosure overcome the above-referenced technical challenges and difficulties, and provide various technical improvements and advantages. For example, various embodiments of the present disclosure provide an example non-invasive breath analysis platform that includes an example non-invasive breath analyzer apparatus. In some embodiments, the example non-invasive breath analyzer apparatus comprises an example image generating device and an example spectrometric data analyzing device.

In some embodiments, the example image generating device generates exhaled breath digital image data objects. For example, laser light that is emitted from an example laser emitting device and travels through exhaled breath can be detected by the example image generating device, and the example image generating device generates exhaled breath digital image data objects based at least in part on the detected laser light. In some embodiments, the laser light can traverse through a large portion of exhaled breath. For example, the exhaled breath may flow in a horizontal direction, and the laser light may traverse in a vertical direction. As such, the exhaled breath digital image data object generated in accordance with some embodiments of the present disclosure can capture accurate representations of exhaled breath, and therefore can provide technical benefits and advantages such as, but not limited to, improving accuracies in analyzing exhaled breath.

In some embodiments, the example spectrometric data analyzing device receives the exhaled breath digital image data object from the example image generating device. In some embodiments, the example spectrometric data analyzing device converts exhaled breath (as represented by the exhaled breath digital image data object) into exhaled breath spectrometric data objects. For example, the exhaled breath spectrometric data objects may comprise photographic metadata and/or spectrometric metadata that can be extracted from the exhaled breath digital image data object. As such, the non-invasive breath analyzer apparatus converts exhaled breath into exhaled breath spectrometric data objects based on laser spectroscopy, overcoming technical challenges and difficulties (e.g., associated with capturing all available data) that are faced by many methods (such as, but not limited to, chemical-solution based methods described above).

In some embodiments, the example spectrometric data analyzing device generates spectrometric-based prediction data objects based on the exhaled breath spectrometric data objects. For example, the example spectrometric data analyzing device provides an example trained machine learning computing model. In such an example, the example trained machine learning computing model receives a plurality of exhaled breath spectrometric data objects as inputs, and the trained machine learning computing model generates spectrometric-based prediction data objects as outputs. In some embodiments, the example spectrometric data analyzing device adjusts the example trained machine learning computing model through training, which can improve accuracies in generating spectrometric-based prediction data objects and overcome technical challenges and difficulties that are plagued by many methods and systems.

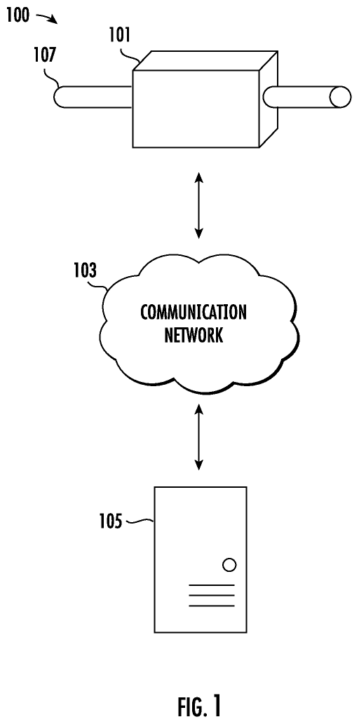

Referring now to FIG. 1, an example schematic diagram in accordance with some embodiments of the present disclosure is provided. In particular, the example schematic diagram illustrates an example non-invasive breath analysis platform 100. In some embodiments, the example non-invasive breath analysis platform 100 enables real-time and/or on-demand remote monitoring of health conditions and/or health levels associated with users (such as, but not limited to, patients).

In the example shown in FIG. 1, the example non-invasive breath analysis platform 100 comprises a non-invasive breath analyzer apparatus 101, a communication network 103, and a non-invasive breath analyzer server 105.

As described above, the example non-invasive breath analysis platform 100 can provide real-time and/or on-demand remote monitoring of health conditions associated with users/patients. For example, the non-invasive breath analyzer apparatus 101 of the example non-invasive breath analysis platform 100 can derive diagnostic information related to health conditions and/or health levels associated with a user and/or a patient based on, for example but not limited to, implementing laser spectroscopy on exhaled breath from users.

In the present disclosure, the term “laser spectroscopy” refers to methods and/or techniques that utilizes deflections/absorptions of laser light caused by a sample (and/or interactions between the laser light and the sample) to determine, analyze, and/or predict the compositions, characteristics, properties, and/or the like associated with the sample. In some embodiments, the sample may be in a gaseous form such as, but not limited to, exhaled breath (for example, exhaled breath captured/collected by an example non-invasive breath analyzer apparatus in accordance with various embodiments of the present disclosure).

As an example, some embodiments of the present disclosure implement laser absorption spectroscopy to determine, analyze, and/or predict the compositions, characteristics, properties, and/or the like that are associated with exhaled breath from users. In such an example, laser light tuned through a wavelength range (for example, generated and emitted by an example laser emitting device described herein) may travel through the exhaled breath. As the laser light travels through the exhaled breath, compounds in the exhaled breath such as, but not limited to, chemicals (including, but not limited to, nitric oxide, monoxide), volatile organic compounds (VOCs), molecules, microorganisms (e.g., bacterial pathogens), viruses, and/or the like may absorb some of the laser light at one or more wavelengths within the wavelength range. In some embodiments, based on the absorption level of the laser light at different wavelengths, the compositions, characteristics, properties, and/or the like of the exhaled breath can be determined, analyzed, and/or predicted.

While the description above provides an example of laser spectroscopy in the form of laser absorption spectroscopy, it is noted that the scope of the present disclosure is not limited to the example above. In some embodiments, an example method may implement one or more additional or alternative forms of laser spectroscopy, such as, but not limited to, laser-induced fluorescence spectroscopy, Raman spectroscopy, and/or the like.

Referring to FIG. 1, for example, the non-invasive breath analyzer apparatus 101 comprises at least one breathing tube 107 that is connected to a non-invasive breath analyzer housing of the non-invasive breath analyzer apparatus 101. In some embodiments, a user (such as, but not limited to, a patient) can breathe into the at least one breathing tube 107, and the exhaled breath from the user (such as, but not limited to, the patient) can be passed along the at least one breathing tube 107 and into the non-invasive breath analyzer housing of the non-invasive breath analyzer apparatus 101. In some embodiments, the at least one breathing tube 107 is disposable.

In some embodiments, at least one laser emitting device and at least one image generating device are positioned within the non-invasive breath analyzer housing of the non-invasive breath analyzer apparatus 101.

In the present disclosure, the term “laser emitting device” refers to an apparatus or a device that emits laser light (for example, but not limited to, laser beams). Examples of laser emitting devices include, but are not limited to, laser diodes (such as, but not limited to, tunable laser diodes, signal-frequency laser diodes, multi-frequency laser diodes, and/or the like), solid-state lasers, and/or the like. In some embodiments, the laser emitting device tunes the wavelengths of the laser light through a wavelength range.

In the present disclosure, the term “image generating device” refers to an apparatus or a device that can generate one or more digital image data objects. For example, an example image generating device may be in the form of or comprise one or more image sensors, one or more cameras, one or more photodetectors, and/or the like.

In the present disclosure, the term “data object” refers to a data structure that represents, provides, and/or describes one or more content, functionalities and/or characteristics associated with data and/or information. In the present disclosure, the term “metadata” refers to a parameter, a data field, a data element, or the like that describes an attribute of a data object.

In the present disclosure, the term “digital image data object” refers to a type of data object that represents, comprises, and/or is associated with one or more digital images. In the present disclosure, the term “exhaled breath digital image data object” refers to a type of digital image data object that represents, comprises, and/or is associated with a digital image of exhaled breath.

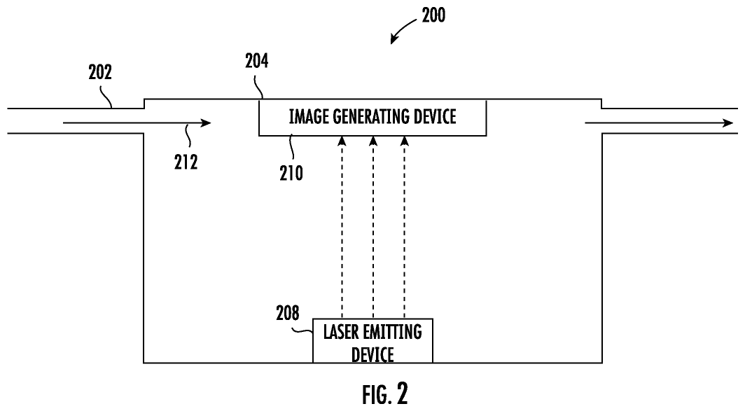

As described above, the laser emitting device may emit laser light (for example, but not limited to, one or more laser beams) through exhaled breath. In some embodiments, the laser emitting device may tune the laser light through a wavelength range. In such embodiments, the image generating device may generate an exhaled breath digital image data object of the exhaled breath as the laser light (for example, but not limited to, one or more laser beams) travels through the exhaled breath and is tuned within the wavelength range. In such embodiments, the exhaled breath digital image data object comprises data and/or information showing deflections/absorptions of the laser light caused by the exhaled breath (and/or interactions between the laser light and the exhaled breath) as the laser light travels through the exhaled breath and is tuned within the wavelength range. Additional details are described herein, including, but not limited to, those described in connection with at least FIG. 2 and FIG. 5 to FIG. 9.

In some embodiments, the non-invasive breath analyzer apparatus 101 comprises a spectrometric data analyzing device. In some embodiments, the spectrometric data analyzing device may be in the form of a computing device that includes a processor (such as, but not limited to, a controller, a central processing unit, and/or the like) and a memory (such as, but not limited to, volatile memory, non-volatile memory, and/or the like). In some embodiments, the spectrometric data analyzing device generates exhaled breath spectrometric data objects.

For example, the at least one image generating device of the non-invasive breath analyzer apparatus 101 is in data communications with the spectrometric data analyzing device. As described in the examples above, in some embodiments, the non-invasive breath analyzer apparatus 101 implements the laser emitting device to emit laser light (for example, but not limited to, one or more laser beams) through the exhaled breath in the non-invasive breath analyzer apparatus 101, and implements the image generating device to generate exhaled breath digital image data objects of the exhaled breath as the laser light (for example, but not limited to, one or more laser beams) travels through the exhaled breath. In some embodiments, the image generating device transmits the exhaled breath digital image data object to the spectrometric data analyzing device.

In some embodiments, the spectrometric data analyzing device generates the exhaled breath spectrometric data objects based at least in part on the exhaled breath digital image data objects, additional details are described herein.

In some embodiments, the spectrometric data analyzing device provides the exhaled breath spectrometric data objects to one or more machine learning computing models, and the one or more machine learning computing models can generate one or more spectrometric-based prediction data objects. In some embodiments, the one or more spectrometric-based prediction data objects can provide predictions on the diagnosis of the user or patient's health conditions that are related to, for example but not limited to, asthma, renal and liver diseases, lung cancer, chronic obstructive pulmonary disease, inflammatory lung disease, metabolic disorders, and/or the like. Additional details are described herein.

In some embodiments, the non-invasive breath analyzer apparatus 101 is in data communications with the non-invasive breath analyzer server 105 via, for example, but not limited to, the communication network 103.

In some embodiments, the communication network 103 may include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, etc.). For example, the communication network 103 may include an 802.11, 802.16, 802.20, and/or WiMAX network. Additionally, or alternatively, the communication network 103 may include a public network (such as the Internet), a private network (such as an intranet), or combinations thereof, and may utilize a variety of networking protocols including, but not limited to, TCP/IP based networking protocols. For instance, the networking protocol may be customized to suit the needs of the non-invasive breath analyzer server 105 and/or the non-invasive breath analyzer apparatus 101. In some embodiments, the protocol is a custom protocol of JSON objects sent via a WebSocket channel. In some embodiments, the protocol is JSON over RPC, JSON over REST/HTTP, and the like.

In some embodiments, the non-invasive breath analyzer server 105 may provide functions such as, but not limited to, data collocation and/or data distribution. For example, the non-invasive breath analyzer server 105 may receive data and/or information from other devices such as, but not limited to, the non-invasive breath analyzer apparatus 101, one or more other network servers, one or more other computing devices (such as, but not limited to, other user devices), and/or the like. Additionally, or alternatively, the non-invasive breath analyzer server 105 may transmit data and/or information to other devices such as, but not limited to, the non-invasive breath analyzer apparatus 101, one or more other network servers, one or more other computing devices (such as, but not limited to, other user devices), and/or the like.

As an example, the non-invasive breath analyzer server 105 may generate and/or update electronic health records (EMRs) associated with users of the example non-invasive breath analysis platform 100. In such an example, the non-invasive breath analyzer server 105 may receive data and/or information such as, but not limited to, spectrometric-based prediction data objects associated with the users from the non-invasive breath analyzer apparatus 101, and may update the EMRs associated with the users based on the spectrometric-based prediction data objects. For example, the non-invasive breath analyzer server 105 may update the EMRs to include one or more predictions, estimates, and/or forecasts from the spectrometric-based prediction data objects.

As another example, the non-invasive breath analyzer server 105 may receive one or more predicted health condition indications and/or one or more predicted condition progression indications from one or more spectrometric data analyzing devices (such as, but not limited to, the spectrometric data analyzing device 101 described above). For example, the spectrometric data analyzing device 101 may generate one or more predicted health condition indications based on the at least one spectrometric-based prediction data object from the at least one trained machine learning computing model. Additionally, or alternatively, the spectrometric data analyzing device 101 may generate one or more predicted condition progression indications based at least in part on comparing spectrometric-based prediction data objects associated with different time points. In some embodiments, the non-invasive breath analyzer server 105 may transmit one or more health alerts and/or diagnosis alerts to one or more user devices associated with the users/patients and/or healthcare providers (such as, but not limited to, physicians, nurses, and/or the like) to help and support patient health diagnosis and/or monitoring. Additional details are described herein.

As illustrated in the various examples in connection with FIG. 1 above, the example non-invasive breath analysis platform 100 implements laser spectrometry based techniques to analyze exhaled breath of a user/patient in a non-invasive way to predict health conditions and/or diseases diagnosis associated with the user/patient (including, but not limited to, those related to asthma, renal and liver diseases, lung cancer, chronic obstructive pulmonary disease, inflammatory lung disease, metabolic disorders, and/or the like). For example, the spectrometric data analyzing device can generate/populate exhaled breath spectrometric data objects based on the digital image of exhaled breath from the users/patients, and can provide exhaled breath spectrometric data objects as inputs to machine learning computing models that generate spectrometric-based prediction data objects that predict the health conditions of users and/or patients and assist in diagnosing health conditions associated with users and/or patients. As such, various embodiments of the present disclosure provide technical advantages and benefits such as, but not limited to, improved accuracies in analyzing exhaled breath from users/patients and improved reliabilities of predicted health conditions of users/patients, additional details of which are described herein.

Referring now to FIG. 2, an example schematic diagram illustrating at least an example portion of an example non-invasive breath analyzer apparatus 200 in accordance with some embodiments of the present disclosure is provided. In some embodiments, the example non-invasive breath analyzer apparatus 200 is the same as or at least similar to the example non-invasive breath analyzer apparatus 101 illustrated and described above in connection with FIG. 1.

Similar to those described above in connection with the example non-invasive breath analyzer apparatus 101 of FIG. 1, the example non-invasive breath analyzer apparatus 200 comprises at least one breathing tube 202 and a breath analyzer housing 204.

In some embodiments, the at least one breathing tube 202 is removably attached to the breath analyzer housing 204. For example, the breath analyzer housing 204 may comprise a breath analyzer housing side surface that defines a breathing tube opening. In such an example, the at least one breathing tube 202 may be attached to the breath analyzer housing 204 where an end of the at least one breathing tube 202 is inserted into the breathing tube opening.

While the example above describes an example breathing tube opening on a breath analyzer housing side surface of the breath analyzer housing, it is noted that the scope of the present disclosure is not limited to the example above. In some examples, the at least one breathing tube 202 may additionally or alternatively be removably attached to one or more other surfaces of the breath analyzer housing.

In some embodiments, when the at least one breathing tube 202 is attached to the breath analyzer housing 204, the at least one breathing tube 202 provides a flow channel so that exhaled breath can flow into the breath analyzer housing 204. For example, a user may blow exhaled breath through the at least one breathing tube 202 into the breath analyzer housing 204, as shown by the arrow 212.

In some embodiments, the at least one breathing tube 202 is disposable and replaceable. For example, after a user blows exhaled breath into the at least one breathing tube 202, the at least one breathing tube 202 can be removed from the breath analyzer housing 204, and a new breathing tube can be inserted into the breathing tube opening of the breath analyzer housing 204 as described above. As such, the disposable and replaceable characteristics of the at least one breathing tube 202 provide technical advantages and benefits such as, but not limited to, reducing or eliminating the likelihood of cross-contaminations between different samples (e.g. different exhaled breath from different users/patients).

In some embodiments, the example non-invasive breath analyzer apparatus 200 comprises a laser emitting device 208 and an image generating device 210.

In some embodiments, the laser emitting device 208 comprises one or more of laser diodes (such as, but not limited to, signal-frequency laser diodes, multi-frequency laser diodes, and/or the like), solid-state lasers, and/or the like, similar to those described above.

In some embodiments, the laser emitting device 208 emits laser light (for example, but not limited to, one or more laser beams). In some embodiments, the laser emitting device 208 is positioned within the breath analyzer housing 204 of the example non-invasive breath analyzer apparatus 200. As described above, the breath analyzer housing 204 of the example non-invasive breath analyzer apparatus 200 receives exhaled breath from a user through the at least one breathing tube 202. In some embodiments, the laser emitting device 208 is disposed within the breath analyzer housing 204 of the example non-invasive breath analyzer apparatus 200 at a location and a position such that the laser light emitted by the laser emitting device 208 can travel through at least some of the exhaled breath.

In the example shown in FIG. 2, the at least one breathing tube 202 is positioned near the top of a side of the breath analyzer housing 204 of the example non-invasive breath analyzer apparatus 200. As such, exhaled breath flows into the breath analyzer housing 204 of the example non-invasive breath analyzer apparatus 200 near the top of the breath analyzer housing 204. In the example shown in FIG. 2, the laser emitting device 208 is secured to a bottom inner surface of the breath analyzer housing 204, and emits laser light (for example, but not limited to, one or more laser beams) towards to the top of the breath analyzer housing 204 of the example non-invasive breath analyzer apparatus 200. As such, the laser light emitted by the laser emitting device 208 can travel through at least some of the exhaled breath within the breath analyzer housing 204 of the example non-invasive breath analyzer apparatus 200.

While the description above provides example locations and positions of at least one breathing tube and the laser emitting device, it is noted that the scope of the present disclosure is not limited to the description above. In some examples, the location(s) and/or the position(s) of the breathing tube and/or the laser emitting device are different from those shown in FIG. 2.

In some embodiments, the breath analyzer housing 204 of the example non-invasive breath analyzer apparatus 200 comprises one or more laser shielding materials that can absorb laser light and/or block laser light from escaping from the breath analyzer housing 204. Examples of laser shielding materials include, but are not limited to, dark colored glasses, acrylic sheets, and/or the like.

In some embodiments, the image generating device 210 comprises one or more image sensors, one or more cameras, one or more photodetectors, and/or the like.

In some embodiments, the image generating device 210 generates digital image data objects. In some embodiments, the image generating device 210 is positioned within the breath analyzer housing 204 of the example non-invasive breath analyzer apparatus 200. As described above, the laser emitting device 208 emits laser light (for example, but not limited to, one or more laser beams) through exhaled breath that is received within the breath analyzer housing 204 of the example non-invasive breath analyzer apparatus 200. In some embodiments, the example image generating device 210 is disposed within the breath analyzer housing 204 of the example non-invasive breath analyzer apparatus 200 at a location and a position such that the digital images generated by the example image generating device 210 capture at least a portion of the exhaled breath after the laser light (for example, but not limited to, one or more laser beams) emitted by the laser emitting device 208 travels through said portion of the exhaled breath.

In the example shown in FIG. 2, the at least one breathing tube 202 is positioned at near the top of a side of the breath analyzer housing 204 of the example non-invasive breath analyzer apparatus 200. The laser emitting device 208 is secured to a bottom inner surface of the breath analyzer housing 204, and emits laser light (for example, but not limited to, one or more laser beams) towards to the top of the breath analyzer housing 204 of the example non-invasive breath analyzer apparatus 200. In some embodiments, the image generating device 210 is secured to a top inner surface of the breath analyzer housing 204, and is position to capture one or more digital images of the exhaled breath from the at least one breathing tube 202 after laser light (for example, but not limited to, one or more laser beams) emitted by the laser emitting device 208 travel through the exhaled breath. In some embodiments, the image generating device 210 is aligned to receive laser light from the laser emitting device 208.

In some embodiments, the image generating device 210 transmits the one or more digital images of exhaled breath to a spectrometric data analyzing device, and the spectrometric data analyzing device converts the digital images of the exhaled breath into exhaled breath spectrometric data objects, details of which are described herein. In some embodiments, the spectrometric data analyzing device further implements at least one trained machine learning computing model to analyze the exhaled breath spectrometric data objects and generate at least one spectrometric-based prediction data object, details of which are described herein. In some embodiments, the spectrometric data analyzing device further transmits the at least one spectrometric-based prediction data object to a non-invasive breath analyzer server (such as, but not limited to, the non-invasive breath analyzer server 105 described above in connection with FIG. 1), which can provide support and assistance in helping physicians diagnose health conditions associated with users/patients, details of which are described herein.

In some embodiments, the example non-invasive breath analyzer apparatus 200 may provide various operation modes that include, but not limited to, a real-time analysis mode and an on-demand analysis mode. In some embodiments, a user of the example non-invasive breath analyzer apparatus 200 may choose the operation mode based on the user's need.

For example, when a user chooses to operate the example non-invasive breath analyzer apparatus 200 in the real-time analysis mode, the user may continuously provide exhaled breath to the example non-invasive breath analyzer apparatus 200 through the at least one breathing tube 202. For example, the at least one breathing tube 202 may be connected to a breathing mask that the user is wearing. In such an example, the laser emitting device 208 of the example non-invasive breath analyzer apparatus 200 may emit laser light (for example, but not limited to, one or more laser beams) at certain time intervals, and the image generating device 210 of the example non-invasive breath analyzer apparatus 200 may capture digital images at these time intervals and generate digital image data objects. In some embodiments, the spectrometric data analyzing device of the example non-invasive breath analyzer apparatus 200 converts digital image data objects of the exhaled breath into exhaled breath spectrometric data objects, generates spectrometric-based prediction data objects by providing the exhaled breath spectrometric data objects to one or more machine learning computing models, and transmits the spectrometric-based prediction data objects to non-invasive breath analyzer servers. As such, under real-time analysis mode, the example non-invasive breath analyzer apparatus 200 can provide real-time analysis of exhaled breath of users by implementing laser spectroscopy techniques, which in turn can provide support and assistance to physicians in helping them diagnose health conditions of the users/patients.

As another example, when a user chooses to operate the example non-invasive breath analyzer apparatus 200 in the on-demand analysis mode, the laser emitting device 208, the image generating device 210, and the spectrometric data analyzing device of the example non-invasive breath analyzer apparatus 200 may be activated when the user provides a user input to operate (for example, via one or more input/output circuits such as, but not limited to, keyboards, touch screens, and/or the like). As an example, the spectrometric data analyzing device of the example non-invasive breath analyzer apparatus 200 may comprise a touch screen that displays user interface elements (for example, an operation mode selection menu with options that include the real-time analysis mode and the on-demand analysis mode).

In some embodiments, after the user chooses the on-demand analysis mode and confirms starting analysis via one or more input/output circuits, the spectrometric data analyzing device of the example non-invasive breath analyzer apparatus 200 may transmit activation signals to the laser emitting device 208 and the image generating device 210, so that the laser emitting device 208 emits laser light (for example, but not limited to, one or more laser beams), the image generating device 210 generates one or more digital image data objects of exhaled breath, and the spectrometric data analyzing device generates at least one spectrometric-based prediction data object. After the user requests and confirms stopping analysis by providing inputs via one or more input/output circuits, the spectrometric data analyzing device of the example non-invasive breath analyzer apparatus 200 may transmit deactivation signals to the laser emitting device 208 and to the image generating device 210. As such, the on-demand analysis mode can provide technical benefits and advantages such as, but not limited to, preserving power of the example non-invasive breath analyzer apparatus 200.

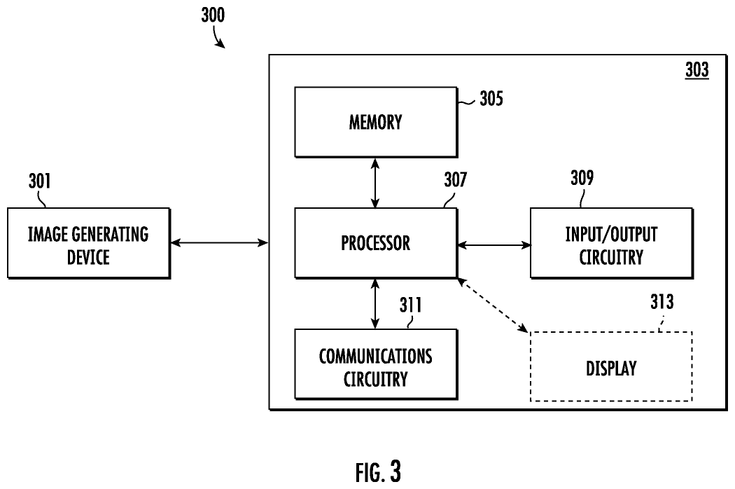

Referring now to FIG. 3, an example block diagram illustrating an example non-invasive breath analyzer apparatus 300 in accordance with some embodiments of the present disclosure is provided. In particular, FIG. 3 illustrates an example image generating device 301 and an example spectrometric data analyzing device 303 in accordance with some embodiments of the present disclosure.

Similar to those described above, the example image generating device 301 may comprise one or more image sensors, one or more cameras, one or more photodetectors, and/or the like that generate one or more digital image data objects (such as, but not limited to, exhaled breath digital image data objects).

In some embodiments, the example spectrometric data analyzing device 303 is in electronic communication with the example image generating device 301 and receives the one or more digital image data objects (such as, but not limited to, exhaled breath digital image data objects) from the example image generating device 301. Similar to those described above, the example spectrometric data analyzing device 303 in accordance with some embodiments of the present disclosure may include one or more computing systems. In the example shown in FIG. 3, the spectrometric data analyzing device 303 comprises a processor 307, a memory 305, an input/output circuitry 309, a communications circuitry 311, and/or a display 313.

In some embodiments, the processor 307 (and/or co-processor or any other processing circuitry assisting or otherwise associated with the processor) may be in communication with the memory 305 via a bus for passing information among components of the apparatus. The memory 305 may be non-transitory and may include, for example, one or more volatile and/or non-volatile memories. In other words, for example, the memory 305 may be an electronic storage device (e.g., a computer readable storage medium). The memory 305 may be configured to store information, data, content, applications, instructions, and/or the like, for enabling the spectrometric data analyzing device 303 to carry out various functions in accordance with example embodiments of the present disclosure.

In some embodiments, the processor 307 may be embodied in a number of different ways and may, for example, include one or more processing devices configured to perform independently. Additionally, or alternatively, the processor 307 may include one or more processors configured in tandem via a bus to enable independent execution of instructions, pipelining, and/or multithreading.

For example, the processor 307 may be embodied as one or more complex programmable logic devices (CPLDs), microprocessors, multi-core processors, co-processing entities, application-specific instruction-set processors (ASIPs), and/or controllers. Further, the processor 307 may be embodied as one or more other processing devices or circuitry. The term circuitry may refer to an entirely hardware embodiment or a combination of hardware and computer program products. Thus, the processor 307 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processor 307 may be configured for a particular use or configured to execute instructions stored in volatile or non-volatile media or otherwise accessible to the processor 307. As such, whether configured by hardware or computer program products, or by a combination thereof, the processor 307 may be capable of performing steps or operations according to embodiments of the present invention when configured accordingly.

The use of the terms “processing circuitry” or “processor” may be understood to include a single core processor, a multi-core processor, multiple processors internal to the apparatus, and/or remote or “cloud” processors.

In some embodiments, the memory 305 stores non-transitory program codes or non-transitory program instructions. In some embodiments, the memory 305 may comprise volatile storage or memory such as, but not limited to, random-access memory (RAM), dynamic random access memory (DRAM), static random access memory (SRAM), fast page mode dynamic random access memory (FPM DRAM), extended data out DRAM (EDO DRAM), synchronous DRAM (SDRAM), double data rate SDRAM (DDR SDRAM), double data rate 2 SDRAM (DDR2 SDRAM), double data rate 3 SDRAM (DDR3 SDRAM), Rambus DRAM (RDRAM), Rambus inline memory module (RIMM), dual in-line memory module (DIMM), single in-line memory module (SIMM), video random access memory (VRAM), cache memory, register memory, and/or the like. Additionally, or alternatively, the memory 305 may comprise non-volatile storage or memory such as, but not limited to, hard disks, read-only memory (ROM), programmable ROM (PROM), erasable programmable ROM (EPROM), electrically erasable programmable ROM (EEPROM), flash memory, Microsoft® management consoles (MMCs), SD memory cards, Memory Sticks, conductive-bridging RAM (CBRAM), parameter RAM (PRAM), ferroelectric RAM (FeRAM), resistive RAM (RRAM), SONOS, racetrack memory, and/or the like. Additionally, or alternatively, the memory 305 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

In some embodiments, the processor 307 may be configured to execute instructions stored in the memory 305 or otherwise accessible to the processor. Alternatively, or additionally, the processor 307 may be configured to execute hard-coded functionality. As such, whether configured by hardware or software methods, or by a combination thereof, the processor 307 may represent an entity (e.g., physically embodied in circuitry) capable of performing operations according to an embodiment of the present disclosure while configured accordingly. Additionally, or alternatively, when the processor 307 is embodied as an executor of software instructions, the instructions may specifically configure the processor to perform the algorithms and/or operations described herein when the instructions are executed.

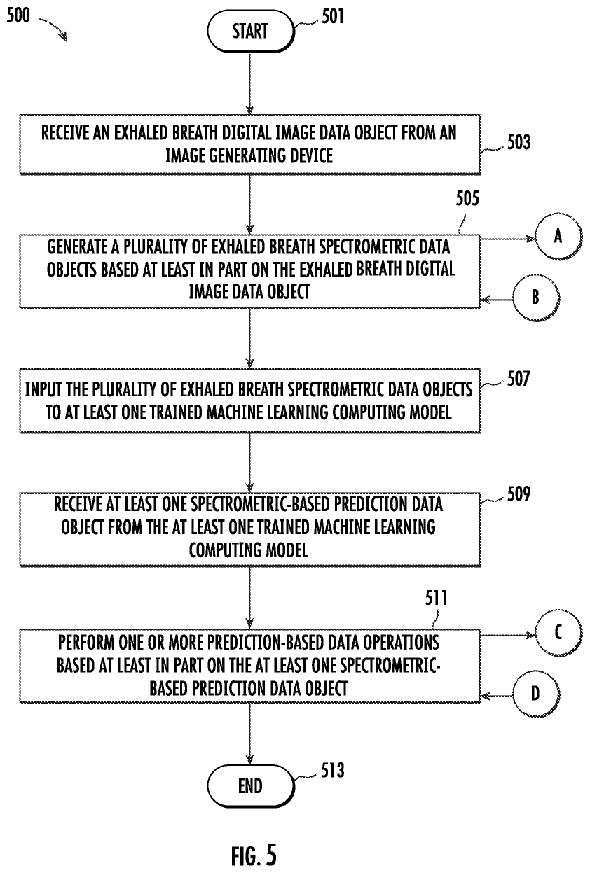

In some embodiments, the memory 305 and the non-transitory program code are configured to, with the processor 307, cause the spectrometric data analyzing device 303 to execute one or more methods and/or operations of method(s) described above with respect to FIG. 1 and FIG. 2 and below with respect to FIG. 5 to FIG. 9. Although the components are described with respect to functional limitations, it should be understood that the particular implementations necessarily include the use of particular hardware. It should also be understood that certain of the components described herein may include similar or common hardware. For example, two sets of circuitries may both leverage use of the same processor, network interface, storage medium, or the like to perform their associated functions, such that duplicate hardware is not required for each set of circuitries. The use of the term “circuitry” as used herein with respect to components of the apparatus should therefore be understood to include particular hardware configured to perform the functions associated with the particular circuitry as described herein.

In some embodiments, the spectrometric data analyzing device 303 may include the input/output circuitry 309 that may, in turn, be in communication with the processor 307 to provide output to the user and, in some embodiments, to receive an indication of a user input. The input/output circuitry 309 may comprise a user interface circuitry and may include a display, which may comprise a web user interface, a mobile application, a user computing device, a kiosk, or the like. In some embodiments, the input/output circuitry 309 may also include a keyboard, a mouse, a joystick, a touch screen, touch areas, soft keys, a microphone, a speaker, or other input/output mechanisms. The processor and/or user interface circuitry comprising the processor may be configured to control one or more functions of one or more user interface elements through computer program instructions (e.g., software and/or firmware) stored on a memory accessible to the processor (e.g., the memory 305, and/or the like).

In some embodiments, the spectrometric data analyzing device 303 may include the display 313 that may, in turn, be in communication with the processor 307 to display renderings of user interface elements. In various examples of the present disclosure, the display 313 may include a liquid crystal display (LCD), a light-emitting diode (LED) display, a plasma (PDP) display, a quantum dot (QLED) display, and/or the like.

In some embodiments, the communications circuitry 311 may be any means such as a device or circuitry embodied in either hardware or a combination of hardware and software that is configured to receive and/or transmit data from/to a network and/or any other device, circuitry, or module in communication with the spectrometric data analyzing device 303. In this regard, the communications circuitry 311 may include, for example, a network interface for enabling communications with a wired or wireless communication network. For example, the communications circuitry 311 may include one or more network interface cards, antennae, buses, switches, routers, modems, and supporting hardware and/or software, or any other device suitable for enabling communications via a network. Additionally, or alternatively, the communication interface may include the circuitry for interacting with the antenna(s) to cause transmission of signals via the antenna(s) or to handle receipt of signals received via the antenna(s).

It is also noted that all or some of the information discussed herein can be based on data that is received, generated and/or maintained by one or more components of example non-invasive breath analyzer 300. In some embodiments, one or more external systems (such as a remote cloud computing and/or data storage system) may also be leveraged to provide at least some of the functionality discussed herein.

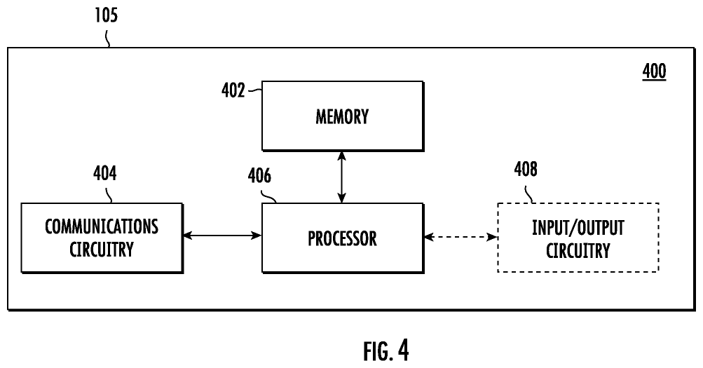

In some embodiments, an example non-invasive breath analyzer server in accordance with some embodiments of the present disclosure (such as, but not limited to, the example non-invasive breath analyzer server 105 shown in FIG. 1) may be embodied by one or more computing systems, such as apparatus 400 shown in FIG. 4.

The apparatus 400 may include a processor 406, a memory 402, an input/output circuitry 408, and a communications circuitry 404. The apparatus 400 may be configured to execute one or more methods and/or operations of method(s) described above with respect to FIG. 1 and FIG. 2 and below with respect to FIG. 5 to FIG. 9. Although these components 402, 404, 406, and 408 are described with respect to functional limitations, it should be understood that the particular implementations necessarily include the use of particular hardware. It should also be understood that certain of these components 402, 404, 406, and 408 may include similar or common hardware. For example, two sets of circuitries may both leverage use of the same processor, network interface, storage medium, or the like to perform their associated functions, such that duplicate hardware is not required for each set of circuitries.

In some embodiments, the processor 406 (and/or co-processor or any other processing circuitry assisting or otherwise associated with the processor) may be in communication with the memory 402 via a bus for passing information among components of the apparatus. The memory 402 is non-transitory and may include, for example, one or more volatile and/or non-volatile memories. In other words, for example, the memory 402 may be an electronic storage device (e.g., a computer-readable storage medium). The memory 402 may be configured to store information, data, content, applications, instructions, or the like for enabling the apparatus to carry out various functions in accordance with example embodiments of the present disclosure.

In some embodiments, the processor 406 may be embodied in a number of different ways and may, for example, include one or more processing devices configured to perform independently. In some examples, the processor 406 may include one or more processors configured in tandem via a bus to enable independent execution of instructions, pipelining, and/or multithreading.

As will be understood, the processor 406 may be embodied in a number of different ways. For example, the processor 406 may be embodied as one or more CPLDs, microprocessors, multi-core processors, co-processing entities, ASIPs, and/or controllers. Further, the processor 406 may be embodied as one or more other processing devices or circuitry. The term circuitry may refer to an entirely hardware embodiment or a combination of hardware and computer program products. Thus, the processor 406 may be embodied as integrated circuits, ASICs, FPGAs, PLAs, hardware accelerators, other circuitry, and/or the like. As will therefore be understood, the processor 406 may be configured for a particular use or configured to execute instructions stored in volatile or non-volatile media or otherwise accessible to the processor 406. As such, whether configured by hardware or computer program products, or by a combination thereof, the processor 406 may be capable of performing steps or operations according to embodiments of the present invention when configured accordingly.

In some embodiments, the memory 402 stores non-transitory program codes or non-transitory program instructions. In some embodiments, the memory 402 may comprise volatile storage or memory such as, but not limited to, RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. Additionally, or alternatively, the memory 402 may comprise non-volatile storage or memory such as, but not limited to, hard disks, ROM, PROM, EPROM, EEPROM, flash memory, MMCs, SD memory cards, Memory Sticks, CBRAM, PRAM, FeRAM, RRAM, SONOS, racetrack memory, and/or the like. Additionally, or alternatively, the memory 402 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

In some embodiments, the processor 406 may be configured to execute instructions stored in the memory 402 or otherwise accessible to the processor 406. In some examples, the processor 406 may be configured to execute hard-coded functionalities. As such, whether configured by hardware or software methods, or by a combination thereof, the processor 406 may represent an entity (e.g., physically embodied in circuitry) capable of performing operations according to an embodiment of the present disclosure while configured accordingly. Alternatively, as another example, when the processor 406 is embodied as an executor of software instructions, the instructions may specifically configure the processor 406 to perform the algorithms and/or operations described herein when the instructions are executed.

In some embodiments, the apparatus 400 may include the input/output circuitry 408 that may, in turn, be in communication with the processor 406 to provide output to the user and, in some embodiments, to receive an indication of a user input. The input/output circuitry 408 may comprise a user interface circuitry and may include a display, which may comprise a web user interface, a mobile application, a user computing device, a kiosk, or the like. In some embodiments, the input/output circuitry 408 may also include a keyboard, a mouse, a joystick, a touch screen, touch areas, soft keys, a microphone, a speaker, or other input/output mechanisms. The processor and/or user interface circuitry comprising the processor may be configured to control one or more functions of one or more user interface elements through computer program instructions (e.g., software and/or firmware) stored on a memory accessible to the processor (e.g., the memory 402, and/or the like).

In some embodiments, the communications circuitry 404 may be any means such as a device or circuitry embodied in either hardware or a combination of hardware and software that is configured to receive and/or transmit data from/to a network and/or any other device, circuitry, or module in communication with the apparatus 400. In this regard, the communications circuitry 404 may include, for example, a network interface for enabling communications with a wired or wireless communication network (such as the communication network described above in connection with FIG. 1). For example, the communications circuitry 404 may include one or more network interface cards, antennae, buses, switches, routers, modems, and supporting hardware and/or software, or any other device suitable for enabling communications via a network. Additionally, or alternatively, the communications circuitry 404 may include the circuitry for interacting with the antenna/antennae to cause transmission of signals via the antenna/antennae or to handle receipt of signals received via the antenna/antennae.

It is also noted that all or some of the information discussed herein can be based on data that is received, generated and/or maintained by one or more components of apparatus 400. In some embodiments, one or more external systems (such as a remote cloud computing and/or data storage system) may also be leveraged to provide at least some of the functionality discussed herein.

In some embodiments, other elements of the apparatus 400 may provide or supplement the functionality of particular circuitry. For example, the processor 406 may provide processing functionality, the memory 402 may provide storage functionality, the communications circuitry 404 may provide network interface functionality, and the like. As will be appreciated, any such computer program instructions and/or other type of code may be loaded onto a computer, processor or other programmable apparatus's circuitry to produce a machine, such that the computer, processor or other programmable circuitry that execute the code on the machine creates the means for implementing various functions, including those described herein.

Various methods described herein, including but not limited to example methods as shown in FIG. 5 to FIG. 9, may provide technical improvements in predicting diseases based on exhale breath from a user.

It is noted that each block of the flowchart, and combinations of blocks in the flowchart, may be implemented by various means such as hardware, firmware, circuitry and/or other devices associated with execution of software including one or more computer program instructions. For example, one or more of the procedures described in FIG. 5 to FIG. 9 may be embodied by computer program instructions, which may be stored by a non-transitory memory of an apparatus employing an embodiment of the present disclosure and executed by a processor in the apparatus. These computer program instructions may direct a computer or other programmable apparatus to function in a particular manner, such that the instructions stored in the computer-readable storage memory produce an article of manufacture, the execution of which implements the function specified in the flowchart block(s).

As described above and as will be appreciated based on this disclosure, embodiments of the present disclosure may be configured as methods, mobile devices, backend network devices, and the like. Accordingly, embodiments may comprise various means including entirely of hardware or any combination of software and hardware. Furthermore, embodiments may take the form of a computer program product on at least one non-transitory computer-readable storage medium having computer-readable program instructions (e.g., computer software) embodied in the storage medium. Similarly, embodiments may take the form of a computer program code stored on at least one non-transitory computer-readable storage medium. Any suitable computer-readable storage medium may be utilized including non-transitory hard disks, CD-ROMs, flash memory, optical storage devices, or magnetic storage devices.

Referring now to FIG. 5, an example method 500 is illustrated. In particular, the example method 500 illustrates examples of generating spectrometric-based prediction data objects in accordance with some embodiments of the present disclosure.

In the example shown in FIG. 5, the example method 500 starts at block 501 and then proceeds to step/operation 503. At step/operation 503, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) receives an exhaled breath digital image data object from an image generating device.

As described above, an example non-invasive breath analyzer apparatus comprises a laser emitting device and an image generating device. In some embodiments, the example non-invasive breath analyzer apparatus receives exhaled breath from a user (for example, via at least one breathing tube). In some embodiments, the laser emitting device emits laser light (for example, but not limited to, one or more laser beams) through the exhaled breath. In some embodiments, the image generating device generates exhaled breath digital image data objects that comprises data and/or information associated with deflections/absorption of the laser light caused by the exhaled breath and/or interactions between the laser light and the exhaled breath as the laser light travels through the exhaled breath and tuned within a wavelength range.

In some embodiments, the image generating device may be in electronic communication with a spectrometric data analyzing device that comprises a processing circuitry, and may transmit exhaled breath digital image data objects to the processing circuitry of the spectrometric data analyzing device.

Referring back to FIG. 5, subsequent and/or in response to step/operation 503, the example method 500 proceeds to step/operation 505. At step/operation 505, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) generates a plurality of exhaled breath spectrometric data objects based at least in part on the exhaled breath digital image data object.

In the present disclosure, the term “exhaled breath spectrometric data object” refers to a type of data object that comprises spectrometric data and/or information associated with exhaled breath.

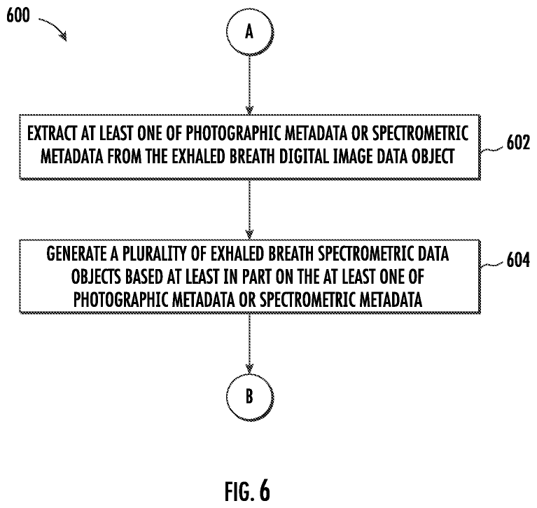

As described above, example embodiments of the present disclosure may implement laser spectroscopy to determine, analyze, and/or predict the compositions, characteristics, properties, and/or the like associated with exhaled breath. For example, the laser emitting device may emit laser light that is tuned through a wavelength range, and the image generating device may generate exhaled breath digital image data objects showing deflections/absorptions of the laser light caused by exhaled breath as the laser light travels through the exhaled breath and is tuned within the wavelength range. In some embodiments, the exhaled breath spectrometric data object may comprise spectrometric data and/or information that represents, indicates, and/or is associated with deflection/absorption levels of the laser light caused by the exhaled breath at various wavelengths within the wavelength range. In some embodiments, the processing circuitry may extract at least one of photographic metadata or spectrometric metadata from the exhaled breath digital image data object received at step/operation 503 to generate the plurality of exhaled breath spectrometric data objects, additional details of which are described in connection with at least FIG. 6.

While the description above provides example spectrometric data and/or information associated with exhaled breath that is represented by, indicated by, and/or associated with an example exhaled breath spectrometric data object, it is noted that the scope of the present disclosure is not limited to the example above. In some embodiments, an example exhaled breath spectrometric data object may comprise spectrometric data and/or information based on, for example, laser-induced fluorescence spectroscopy, Raman spectroscopy, and/or the like.

Referring back to FIG. 5, subsequent and/or in response to step/operation 505, the example method 500 proceeds to step/operation 507. At step/operation 507, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) inputs the plurality of exhaled breath spectrometric data objects to at least one trained machine learning computing model.

In the present disclosure, the term “machine learning computing model” refers to a computer model embedded in, installed on, and/or executed by one or more computing devices such as, but not limited to, spectrometric data analyzing devices, non-invasive breath analyzer servers, and/or the like for generating one or more data objects as outputs in response to receiving one or more data objects as inputs. In some embodiments, the one or more data objects generated by the machine learning computing model comprise, represent, and/or are associated with one or more predictions, estimates, forecasts, and/or the like.

As an example, an example machine learning computing model may be in the form of a computer-executable algorithm that is executed by an example spectrometric data analyzing device. In such an example, the example spectrometric data analyzing device provides exhaled breath spectrometric data objects as inputs to the example machine learning computing model, and the example machine learning computing model generates spectrometric-based prediction data objects as outputs.

Additionally, or alternatively, an example machine learning computing model may be in the form of a computer-executable algorithm that is executed by a non-invasive breath analyzer server. In such an example, the non-invasive breath analyzer server provides exhaled breath spectrometric data objects (for example, from an example spectrometric data analyzing device) as inputs to the example machine learning computing model, and the example machine learning computing model generates spectrometric-based prediction data objects as outputs.

In some embodiments, an example machine learning computing model may comprise an example classification-based estimation model. In the present disclosure, the term “classification-based estimation model” refers to a machine learning computing model that generates predictions, estimations, forecasts on classifications and/or categories of data objects that are provided to the classification-based estimation model as inputs. Examples of classification-based estimation models include, but are not limited to, random forest, Naive Bayes, logistic regression, decision tree, gradient-boosted tree, and/or the like.

While the description above provides an example classification-based estimation model as an example machine learning computing model, it is noted that the scope of the present disclosure is not limited to the description above. In some examples, an example machine learning computing model may comprise one or more additional and/or alternative machine learning models.

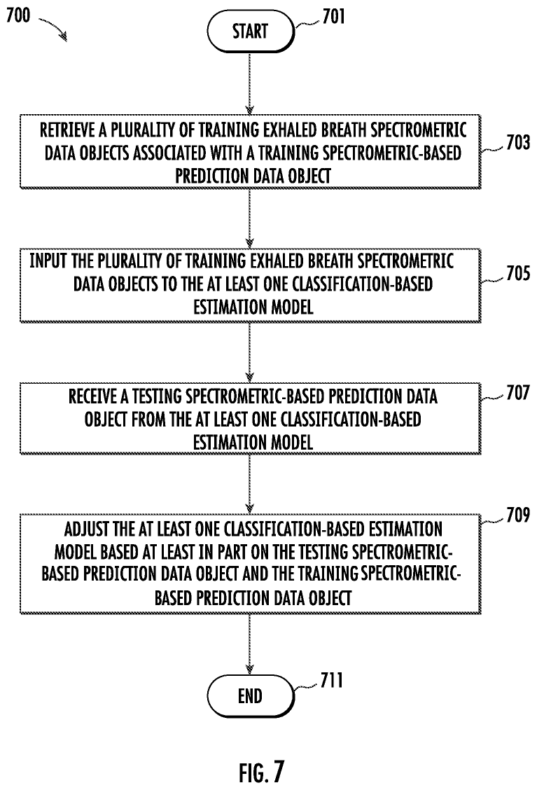

In some embodiments, in order to generate spectrometric-based prediction data objects, a machine learning computing model is trained based on a plurality of training exhaled breath spectrometric data objects associated with a training spectrometric-based prediction data object. In the present disclosure, a machine learning computing model that has been trained is also referred to as a “trained machine learning computing model.” Additional details associated with training the machine learning computing model are described herein, including, but not limited to, those described in connection with at least FIG. 7.

Referring back to FIG. 5, subsequent and/or in response to step/operation 507, the example method 500 proceeds to step/operation 509. At step/operation 509, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) receives at least one spectrometric-based prediction data object from the at least one trained machine learning computing model.

In the present disclosure, the term “spectrometric-based prediction data object” refers to a type of data object that represents, indicates, stores and/or comprises data and/or information associated with one or more predictions, estimates, and/or forecasts on one or more health conditions associated with a user that are based at least in part on spectrometric data and/or information associated with exhaled breath of the user (for example, based on the exhaled breath digital image data object).

For example, an example spectrometric-based prediction data object in accordance with some embodiments of the present disclosure may indicate a prediction, an estimate, and/or a forecast of a disease diagnosis associated with a user based at least in part on the exhaled breath spectrometric data objects associated with exhaled breath of the user. For example, the example spectrometric-based prediction data object may indicate a likelihood that the user has one or more diseases such as, but not limited to, asthma, renal and liver diseases, lung cancer, chronic obstructive pulmonary disease, inflammatory lung disease, metabolic disorders, and/or the like.

Additionally, or alternatively, an example spectrometric-based prediction data object in accordance with some embodiments of the present disclosure may indicate a prediction, an estimate, and/or a forecast of a health condition measurement associated with a user based at least in part on the exhaled breath spectrometric data objects associated with exhaled breath from the user. For example, the health condition measurement may be in the form of a measurement of the amount of VOCs in the exhaled breath. In such an example, the example spectrometric-based prediction data object indicates a prediction, an estimate, and/or a forecast of the amount of VOCs in the exhaled breath. Additionally, or alternatively, the example spectrometric-based prediction data object may indicate a prediction, an estimate, and/or a forecast of other health condition measurements in accordance with some embodiments of the present disclosure.

While the description above provides example predictions, estimates, and/or forecasts associated with example spectrometric-based prediction data objects in accordance with some embodiments of the present disclosure, it is noted that the scope of the present disclosure is not limited to the description above. In some examples, an example spectrometric-based prediction data object may comprise one or more additional and/or alternative predictions, estimates, and/or forecasts.

Referring back to FIG. 5, subsequent and/or in response to step/operation 509, the example method 500 proceeds to step/operation 511. At step/operation 511, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) performs one or more prediction-based data operations based at least in part on the at least one spectrometric-based prediction data object.

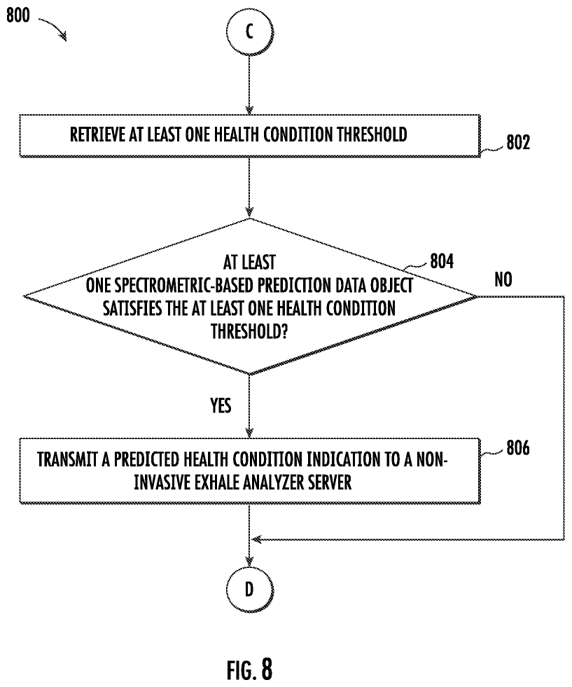

In some embodiments, an example prediction-based data operation may comprise determining whether the at least one spectrometric-based prediction data object received at step/operation 509 satisfies at least one health condition threshold, details of which are described in connection with at least FIG. 8.

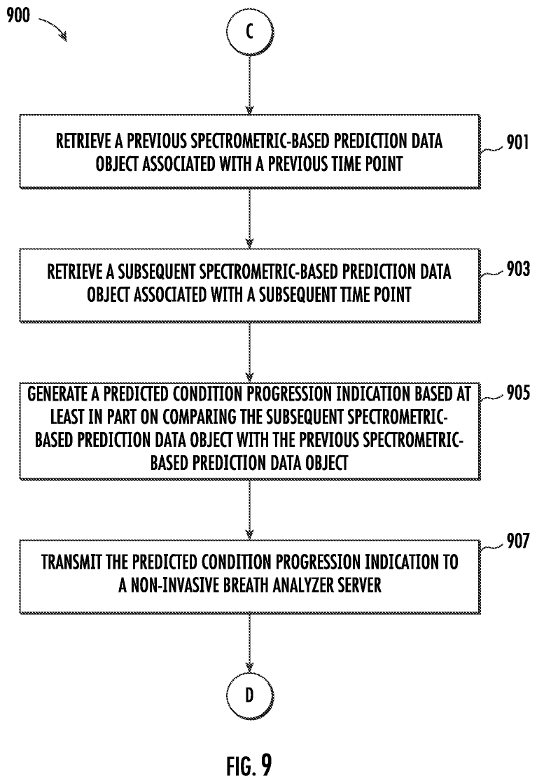

In some embodiments, an example prediction-based data operation may comprise generating and transmitting a predicted condition progression indication based at least in part on the spectrometric-based prediction data objects received at step/operation 509, details of which are described in connection with at least FIG. 9.

While the description above provides examples of prediction-based data operations based on the spectrometric-based prediction data object, it is noted that the scope of the present disclosure is not limited to the description above. In some examples, the processing circuitry may perform one or more prediction-based data operations based at least in part on the at least one spectrometric-based prediction data object in addition to and/or in alternative of the examples described above.

Referring back to FIG. 5, subsequent and/or in response to step/operation 511, the example method 500 proceeds to block 513 and ends.

Referring now to FIG. 6, an example method 600 is illustrated. In particular, the example method 600 illustrates examples of generating exhaled breath spectrometric data objects based on the exhaled breath digital image data objects in accordance with some embodiments of the present disclosure.

In the example shown in FIG. 6, the example method 600 starts at block A. As shown in FIG. 5, block A connects from step/operation 505 where the processing circuitry generates a plurality of exhaled breath spectrometric data objects based at least in part on the exhaled breath digital image data object. As such, the example method 600 illustrated in FIG. 6 provides an example of generating exhaled breath spectrometric data objects based at least in part on the exhaled breath digital image data object.

Referring back to FIG. 6, subsequent to block A, the example method 600 proceeds to step/operation 602. At step/operation 602, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) extracts at least one of photographic metadata or spectrometric metadata from the exhaled breath digital image data object.

In the present disclosure, the “photographic metadata” refers to a type of metadata associated with the exhaled breath digital image data object that indicates, represents, and/or is associated with one or more picture elements or pixels of the exhaled breath digital image data object.

As described above, the exhaled breath digital image data object may represent, comprise, and/or be associated with a digital image of exhaled breath as laser light travels through the exhaled breath. In some embodiments, the photographic metadata of the exhaled breath digital image data object may represent light intensity levels (or gray levels) at various areas on the digital image as the laser light travels through the exhaled breath.

While the description above provides example data and/or information that is represented by photographic metadata, it is noted that the scope of the present disclosure is not limited to the description above. In some examples, example photographic metadata may comprise additional and/or alternative data and/or information.

In the present disclosure, the term “spectrometric metadata” refers to a type of metadata associated with the exhaled breath digital image data object that indicates, represents, and/or is associated with spectrometric data and/or information.

As described above, an example laser emitting device in accordance with some embodiments of the present disclosure may tune the laser light through a wavelength range while emitting the laser light through the exhaled breath. In some embodiments, the spectrometric metadata of the exhaled breath digital image data object may comprise spectrometric data and/or information indicating deflection/absorption levels of the laser light by the exhaled breath at different wavelengths within the wavelength range.

While the description above provides example data and/or information that is represented by spectrometric metadata, it is noted that the scope of the present disclosure is not limited to the description above. In some examples, example spectrometric metadata may comprise additional and/or alternative data and/or information.

Referring back to FIG. 6, subsequent and/or in response to step/operation 602, the example method 600 proceeds to step/operation 604. At step/operation 604, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) generates a plurality of exhaled breath spectrometric data objects based at least in part on the at least one of photographic metadata or spectrometric metadata.

For example, in some embodiments, the processing circuitry generates the plurality of exhaled breath spectrometric data objects based at least in part on the photographic metadata of the exhaled breath digital image data object.

As described above, the photographic metadata of the exhaled breath digital image data object may indicate, for example but not limited to, light intensity levels (or gray levels) at various areas on the digital image as the laser light travels through the exhaled breath. In such an example, the processing circuitry calculates deflection/absorption levels of the laser light at various areas on the digital image based at least in part on the light intensity levels from the photographic metadata, and generates the spectrometric data and/or information for the exhaled breath spectrometric data objects that represents, indicates, and/or is associated with deflection/absorption levels of the laser light by the exhaled breath.

Additionally, or alternatively, the processing circuitry generates the plurality of exhaled breath spectrometric data objects based at least in part on the spectrometric metadata of the exhaled breath digital image data object.

As described above, the spectrometric metadata of the exhaled breath digital image data object may indicate, for example but not limited to, deflection/absorption levels of the laser light caused by the exhaled breath at different wavelengths within the wavelength range. In some embodiments, the processing circuitry generates the spectrometric data and/or information for the exhaled breath spectrometric data objects that represents, indicates, and/or is associated with deflection/absorption levels of the laser light by the exhaled breath

In some embodiments, each of the plurality of exhaled breath spectrometric data objects is associated with one area on the digital image and comprises spectrometric data and/or information associated with that area (for example, deflections/absorptions of the laser light caused by exhaled breath in that area as the laser light travels through the exhaled breath in that area).

Referring back to FIG. 6, subsequent and/or in response to step/operation 604, the example method 600 proceeds to block B. As shown in FIG. 5, block B connects back to step/operation 505 of FIG. 5.

Referring now to FIG. 7, an example method 700 is illustrated. In particular, the example method 700 illustrates examples of generating at least one trained machine learning computing model that generates spectrometric-based prediction data objects through training a machine learning computing model in accordance with some embodiments of the present disclosure.

In the example shown in FIG. 7, the example method 700 starts at block 701 and then proceeds to step/operation 703. At step/operation 703, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) retrieves a plurality of training exhaled breath spectrometric data objects associated with a training spectrometric-based prediction data object.

In some embodiments, the plurality of training exhaled breath spectrometric data objects are retrieved from a training data object repository. In some embodiments, the training data object repository may be in the form of a database or data store. For example, the processing circuitry may be in electronic communications with the training data object repository, and retrieve the plurality of training exhaled breath spectrometric data objects from the training data object repository.

In the present disclosure, the term “training exhaled breath spectrometric data object” refers to a type of exhaled breath spectrometric data object that is generated and/or stored for the purpose of training machine learning computing models. In some embodiments, each of one or more training exhaled breath spectrometric data objects stored in the training data object repository is associated with a training spectrometric-based prediction data object.

As an example, the training exhaled breath spectrometric data objects may be generated by a processing circuitry based on one or more exhaled breath digital image data objects associated with exhaled breath from a user, and the training spectrometric-based prediction data object represents, indicates, stores and/or comprises data and/or information associated with one or more known health conditions associated with the user. For example, the training spectrometric-based prediction data object may indicate whether the user has one or more diseases such as, but not limited to, asthma, renal and liver diseases, lung cancer, chronic obstructive pulmonary disease, inflammatory lung disease, metabolic disorders, and/or the like. Additionally, or alternatively, the training spectrometric-based prediction data object may indicate one or more known health condition measurements associated with the user. Additionally, or alternatively, the training spectrometric-based prediction data object may indicate one or more other known aspects of health conditions associated with the user.

Referring back to FIG. 7, subsequent and/or in response to step/operation 703, the example method 700 proceeds to step/operation 705. At step/operation 705, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) inputs the plurality of training exhaled breath spectrometric data objects to at least one classification-based estimation model.

In some embodiments, the processing circuitry provides the plurality of training exhaled breath spectrometric data objects as inputs to the at least one classification-based estimation model. As described above, examples of classification-based estimation models include, but are not limited to, random forest, Naive Bayes, logistic regression, decision tree, gradient-boosted tree, and/or the like.

While the description above provides an example of training a classification-based estimation model, it is noted that the scope of the present disclosure is not limited to the description above. In some embodiments, an example method may train one or more additional and/or alternative machine learning computing models to generate spectrometric-based prediction data objects.

Referring back to FIG. 7, subsequent and/or in response to step/operation 705, the example method 700 proceeds to step/operation 707. At step/operation 707, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) receives a testing spectrometric-based prediction data object from the at least one classification-based estimation model.

As described above, the at least one classification-based estimation model generates predictions, estimations, and/or forecasts based on the training exhaled breath spectrometric data objects. For example, the at least one classification-based estimation model can generate at least one testing spectrometric-based prediction data object based on the training exhaled breath spectrometric data objects that are provided as inputs at step/operation 705.

In the present disclosure, the term “testing spectrometric-based prediction data object” refers to a type of spectrometric-based prediction data object that is generated by a machine learning computing model prior to the training of the machine learning computing model being completed. For example, the testing spectrometric-based prediction data object represents, indicates, stores and/or comprises data and/or information associated with one or more predictions, estimates, and/or forecasts on one or more health conditions associated with a user that are generated by an untrained machine learning computing model based on the training exhaled breath spectrometric data objects.

For example, the testing spectrometric-based prediction data object may indicate a likelihood that the user has one or more diseases such as, but not limited to, asthma, renal and liver diseases, lung cancer, chronic obstructive pulmonary disease, inflammatory lung disease, metabolic disorders, and/or the like. Additionally, or alternatively, the testing spectrometric-based prediction data object may indicate a prediction, an estimate, and/or a forecast of a health condition measurement associated with a user. Additionally, or alternatively, the testing spectrometric-based prediction data object may indicate a prediction, an estimate, and/or a forecast of other health condition measurements associated with the user.

Referring back to FIG. 7, subsequent and/or in response to step/operation 707, the example method 700 proceeds to step/operation 709. At step/operation 709, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) adjusts the at least one classification-based estimation model based at least in part on the testing spectrometric-based prediction data object and the training spectrometric-based prediction data object.

As described above, the training spectrometric-based prediction data object represents, indicates, stores and/or comprises data and/or information associated with one or more known health conditions associated with the user, while the testing spectrometric-based prediction data object represents, indicates, stores and/or comprises data and/or information associated with predictions, estimates, and/or forecasts on one or more health conditions associated with the user that are generated by the at least one classification-based estimation model. In some embodiments, based on comparing the testing spectrometric-based prediction data object with the training spectrometric-based prediction data object, the processing circuitry may adjust one or more parameters associated with the at least one classification-based estimation model so that the at least one classification-based estimation model can generate testing spectrometric-based prediction data objects providing predictions, estimates, and/or forecasts (for example, related to one or more health conditions) that are close to or match the one or more known health conditions associated with the user.

As an example, the at least one classification-based estimation model may comprise, for example, a random forest model, which constructs one or more decision trees that comprise leaf nodes connected by tree branches. In this example, the random forest inputs the training exhaled breath spectrometric data objects to the one or more decision trees, and the one or more decision trees provide the testing spectrometric-based prediction data object as an output. Continuing in this example, the processing circuitry compares the testing spectrometric-based prediction data object with the training spectrometric-based prediction data object, and adjusts the one or more decision trees (including, but not limited to, adjusting weights of one or more leaf nodes, adjusting connections between leaf nodes, and/or the like) so that health conditions predicted by the testing spectrometric-based prediction data object are close to or match health conditions from the training spectrometric-based prediction data object.

While the description above provides an example of a random forest model as a classification-based estimation model, it is noted that the scope of the present disclosure is not limited to the description above. In some examples, other types of classification-based estimation models and/or other machine learning computing models can be trained based at least in part on the example method 700 illustrated in connection with FIG. 7.

Referring back to FIG. 7, subsequent and/or in response to step/operation 709, the example method 700 proceeds to block 711 and ends.

Referring now to FIG. 8, an example method 800 is illustrated. In particular, the example method 800 illustrates examples of performing prediction-based data operations based at least in part on the spectrometric-based prediction data object(s) (for example, spectrometric-based prediction data object(s) that are generated based at least in part on the example method 500, and/or the example method 600 described above). In particular, the example prediction-based data operation illustrated in FIG. 8 is in the form of determining predicted health condition indications in accordance with some embodiments of the present disclosure.

In the example shown in FIG. 8, the example method 800 starts at block C and then proceeds to step/operation 802. At step/operation 802, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) retrieves at least one health condition threshold.

In the present disclosure, the term “health condition threshold” refers to a threshold value or a threshold parameter associated with the spectrometric-based prediction data object.

For example, the example spectrometric-based prediction data object may indicate a likelihood that the user has one or more diseases such as, but not limited to, asthma, renal and liver diseases, lung cancer, chronic obstructive pulmonary disease, inflammatory lung disease, metabolic disorders, and/or the like. In such an example, the health condition threshold may indicate a threshold value associated with the likelihood of the user having one or more diseases (as indicated by the example spectrometric-based prediction data object). For example, if the likelihood satisfies the threshold value, the processing circuitry determines that the user is predicted to have one or more diseases.

Additionally, or alternatively, the example spectrometric-based prediction data object may indicate a prediction, an estimate, and/or a forecast of a health condition measurement associated with a user. In such an example, the health condition threshold may indicate a threshold value associated with the predicted health condition measurement indicated by the example spectrometric-based prediction data object. For example, if the predicted health condition measurement satisfies the threshold value, the processing circuitry determines that the user is predicted to have one or more diseases.

In some embodiments, an example health condition threshold may be set by a user. In some embodiments, an example health condition threshold may be predetermined by the processing circuitry.

Referring back to FIG. 8, subsequent and/or in response to step/operation 802, the example method 800 proceeds to step/operation 804. At step/operation 804, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) determines whether at least one spectrometric-based prediction data object satisfies at least one health condition threshold.

For example, the processing circuitry compares the likelihood of the user having one or more diseases as indicated by the example spectrometric-based prediction data object with the at least one health condition threshold.

If the likelihood is no less than the threshold value indicated by the at least one health condition threshold, the processing circuitry determines that the at least one spectrometric-based prediction data object satisfies at least one health condition threshold. In other words, the likelihood of the user having one or more diseases equals or is more than the threshold value from the health condition threshold.

If the likelihood is less than the threshold value indicated by the at least one health condition threshold, the processing circuitry determines that the at least one spectrometric-based prediction data object does not satisfy at least one health condition threshold. In other words, the likelihood of the user having one or more diseases is less than the threshold value from the health condition threshold.

Additionally, or alternatively, the processing circuitry compares the predicted health condition measurement indicated by the example spectrometric-based prediction data object with the at least one health condition threshold. If the predicted health condition measurement is no less than the threshold value indicated by the at least one health condition threshold, the processing circuitry determines that the at least one spectrometric-based prediction data object satisfies at least one health condition threshold. If the predicted health condition measurement is less than the threshold value indicated by the at least one health condition threshold, the processing circuitry determines that the at least one spectrometric-based prediction data object does not satisfy at least one health condition threshold.

Referring back to FIG. 8, if, at step/operation 804, the processing circuitry determines that the at least one spectrometric-based prediction data object satisfies at least one health condition threshold, the example method 800 proceeds to step/operation 806. At step/operation 806, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) transmits a predicted health condition indication to a non-invasive exhale analyzer server.

For example, in response to determining that the at least one spectrometric-based prediction data object satisfies the at least one health condition threshold, the processing circuitry determines that the at least one spectrometric-based prediction data object indicates that the user/patient is predicted to have one or more diseases. As such, the processing circuitry generates and transmits a predicted health condition indication to a non-invasive exhale analyzer server, and the predicted health condition indication may comprise, for example but not limited to, a health warning or a disease warning associated with the user/patient.

In some embodiments, subsequent to receiving the predicted health condition indication, the non-invasive exhale analyzer server transmits the predicted health condition indication to other computing device(s) such as, but not limited to, a computing device associated with a physician so that the physician is on alert of the health condition associated with the user.

Referring back to FIG. 8, subsequent to step/operation 806, the example method proceeds to block D, which connects back to step/operation 511 of FIG. 5.

If, at step/operation 804, the processing circuitry determines that the at least one spectrometric-based prediction data object does not satisfy at least one health condition threshold, the example method 800 proceeds to block D, which connects back to step/operation 511 of FIG. 5.

For example, in response to determining that the at least one spectrometric-based prediction data object does not satisfy the at least one health condition threshold, the processing circuitry determines that the at least one spectrometric-based prediction data object does not indicate that the user/patient is predicted to have one or more diseases. As such, the processing circuitry does not generate any health warning or a disease warning associated with the user/patient.

In some embodiments, in response to determining that the at least one spectrometric-based prediction data object does not satisfy the at least one health condition threshold, the processing circuitry may generate and transmit an indication to the non-invasive exhale analyzer server, indicating that the patient is healthy and/or the patient's health condition is stable.

Referring now to FIG. 9, an example method 900 is illustrated. In particular, the example method 900 illustrates examples of performing prediction-based data operations based at least in part on the spectrometric-based prediction data object(s) (for example, spectrometric-based prediction data object(s) that are generated based at least in part on the example method 500 and/or the example method 600 described above). In particular, the example prediction-based data operation illustrated in FIG. 9 is in the form of generating predicted condition progression indications in accordance with some embodiments of the present disclosure.

In the example shown in FIG. 9, the example method 900 starts at block C and then proceeds to step/operation 901. At step/operation 901, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) retrieves a previous spectrometric-based prediction data object associated with a previous time point.

In some embodiments, the previous spectrometric-based prediction data object may be generated in accordance with various example methods described herein, including, but not limited to, the example methods described above in connection with FIG. 5 to FIG. 7.

Referring back to FIG. 9, subsequent and/or in response to step/operation 901, the example method 900 proceeds to step/operation 903. At step/operation 903, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) retrieves a subsequent spectrometric-based prediction data object associated with a subsequent time point.

In some embodiments, the subsequent spectrometric-based prediction data object may be generated in accordance with various example methods described herein, including, but not limited to, the example methods described above in connection with FIG. 5 to FIG. 7.

As described above, the previous spectrometric-based prediction data object is associated with a previous time point, and the subsequent spectrometric-based prediction data object is associated with a subsequent time point. In some embodiments, the previous time point is prior to the subsequent time point.

For example, the previous spectrometric-based prediction data object may be generated based at least in part on an exhaled breath digital image data object that captures exhaled breath from the user at a previous time point, and the subsequent spectrometric-based prediction data object may be generated based at least in part on an exhaled breath digital image data object that captures exhaled breath from the user at a subsequent time point.

Referring back to FIG. 9, subsequent and/or in response to step/operation 903, the example method 900 proceeds to step/operation 905. At step/operation 905, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) generates a predicted condition progression indication based at least in part on comparing the subsequent spectrometric-based prediction data object with the previous spectrometric-based prediction data object.

As described above, the previous spectrometric-based prediction data object is generated based on a previous sample of exhaled breath from a user, and the previous sample of exhaled breath is taken prior to a subsequent sample of exhaled breath (based on which the subsequent spectrometric-based prediction data object is generated). As such, by comparing the subsequent spectrometric-based prediction data object with the previous spectrometric-based prediction data object, the processing circuitry can predict whether there is a progression associated with the one or more health conditions associated with the user/patient and can generate a predicted condition progression indication that indicates whether there is a predicted progression.

For example, the previous spectrometric-based prediction data object may indicate a likelihood that the user has one or more diseases at the previous time point, and the subsequent spectrometric-based prediction data object may indicate a likelihood that the user has one or more diseases at the subsequent time point. In such an example, the processing circuitry may determine whether there is an increase in the likelihood from the previous time point to the subsequent time point. If so, the processing circuitry generates predicted condition progression indication indicating that the user has likely contracted the one or more diseases or that one or more diseases are likely progressing in the user. If not, the processing circuitry generates predicted condition progression indication indicating that the user is unlikely to contract the one or more diseases or one or more diseases are likely diminishing in the user.

Additionally, or alternatively, the previous spectrometric-based prediction data object may indicate a prediction, an estimate, and/or a forecast of a health condition measurement associated with a user at the previous time point, and the subsequent spectrometric-based prediction data object may indicate a prediction, an estimate, and/or a forecast of a health condition measurement associated with a user at the subsequent time point. In such an example, the processing circuitry may determine any change in the health condition measurements from the previous time point to the subsequent time point.

In some embodiments, the processing circuitry compares the change in the health condition measurements with a health condition threshold, similar to those described above in connection with at least FIG. 8. For example, if the processing circuitry determines that the change in the health condition measurements satisfies the health condition threshold, the processing circuitry determines that the comparison between the previous spectrometric-based prediction data object and the previous spectrometric-based prediction data object indicates that the user has likely contracted the one or more diseases or that one or more diseases are likely progressing in the user. If the processing circuitry determines that the change in the health condition measurements does not satisfy the health condition threshold, the processing circuitry determines that the comparison between the previous spectrometric-based prediction data object and the previous spectrometric-based prediction data object indicates that the user is unlikely to contract the one or more diseases or one or more diseases are likely diminishing in the user.

Referring back to FIG. 9, subsequent and/or in response to step/operation 905, the example method 900 proceeds to step/operation 907. At step/operation 907, a processing circuitry (such as, but not limited to, the processor 307 of the spectrometric data analyzing device 303 described above in connection with at least FIG. 1 and FIG. 3, and/or the processor 406 of the non-invasive breath analyzer server 105 described above in connection with at least FIG. 1 and FIG. 4) transmits the predicted condition progression indication to a non-invasive breath analyzer server.

In some embodiments, subsequent to receiving the predicted condition progression indication, the non-invasive exhale analyzer server transmits the predicted condition progression indication to other computing device(s) such as, but not limited to, a computing device associated with a physician, so that the physician is on alert of the health condition associated with the user.

Referring back to FIG. 9, subsequent to step/operation 907, the example method proceeds to block D, which connects back to step/operation 511 of FIG. 5.

It is to be understood that the disclosure is not to be limited to the specific embodiments disclosed, and that modifications and other embodiments are intended to be included within the scope of the appended claims. Although specific terms are employed herein, they are used in a generic and descriptive sense only and not for purposes of limitation, unless described otherwise.

1. A non-invasive breath analyzer apparatus comprising:
an image generating device that is positioned within a breath analyzer housing and generates an exhaled breath digital image data object; and
a spectrometric data analyzing device that is in electronic communication with the image generating device and comprises a processor and a memory storing a non-transitory program code, wherein the memory and the non-transitory program code are configured to, with the processor, cause the spectrometric data analyzing device to:
receive the exhaled breath digital image data object from the image generating device;
generate a plurality of exhaled breath spectrometric data objects based at least in part on the exhaled breath digital image data object;
input the plurality of exhaled breath spectrometric data objects to at least one trained machine learning computing model;
receive at least one spectrometric-based prediction data object from the at least one trained machine learning computing model; and
perform one or more prediction-based data operations based at least in part on the at least one spectrometric-based prediction data object.

2. The non-invasive breath analyzer apparatus of claim 1, wherein, when generating the plurality of exhaled breath spectrometric data objects, the memory and the non-transitory program code are configured to, with the processor, cause the spectrometric data analyzing device to:
extract at least one of photographic metadata or spectrometric metadata from the exhaled breath digital image data object; and
generate the plurality of exhaled breath spectrometric data objects based at least in part on the at least one of photographic metadata or spectrometric metadata.

3. The non-invasive breath analyzer apparatus of claim 1, wherein the at least one trained machine learning computing model comprises at least one trained classification-based estimation model.

4. The non-invasive breath analyzer apparatus of claim 3, wherein, prior to inputting the plurality of exhaled breath spectrometric data objects to the at least one trained machine learning computing model, the memory and the non-transitory program code are configured to, with the processor, cause the spectrometric data analyzing device to:
train at least one classification-based estimation model.

5. The non-invasive breath analyzer apparatus of claim 4, wherein, when training the at least one classification-based estimation model, the memory and the non-transitory program code are configured to, with the processor, cause the spectrometric data analyzing device to:
retrieve a plurality of training exhaled breath spectrometric data objects associated with a training spectrometric-based prediction data object;
input the plurality of training exhaled breath spectrometric data objects to the at least one classification-based estimation model;
receive a testing spectrometric-based prediction data object from the at least one classification-based estimation model; and
adjust the at least one classification-based estimation model based at least in part on the testing spectrometric-based prediction data object and the training spectrometric-based prediction data object.

6. The non-invasive breath analyzer apparatus of claim 1, wherein, when performing the one or more prediction-based data operations based at least in part on the at least one spectrometric-based prediction data object, the memory and the non-transitory program code are configured to, with the processor, cause the spectrometric data analyzing device to:
determine whether the at least one spectrometric-based prediction data object satisfies a health condition threshold; and
in response to determining that the at least one spectrometric-based prediction data object satisfies the health condition threshold, transmit a predicted health condition indication to a non-invasive breath analyzer server.

7. The non-invasive breath analyzer apparatus of claim 1, wherein, when performing the one or more prediction-based data operations based at least in part on the at least one spectrometric-based prediction data object, the memory and the non-transitory program code are configured to, with the processor, cause the spectrometric data analyzing device to:
retrieve a previous spectrometric-based prediction data object associated with a previous time point;
retrieve a subsequent spectrometric-based prediction data object associated with a subsequent time point; and
generate a predicted condition progression indication based at least in part on comparing the subsequent spectrometric-based prediction data object with the previous spectrometric-based prediction data object.

8. A computer program product for non-invasive breath analysis, the computer program product comprising at least one non-transitory computer-readable storage medium having computer-readable program code portions stored therein, the computer-readable program code portions comprising an executable portion configured to:
receive an exhaled breath digital image data object from an image generating device;
generate a plurality of exhaled breath spectrometric data objects based at least in part on the exhaled breath digital image data object;
input the plurality of exhaled breath spectrometric data objects to at least one trained machine learning computing model;
receive at least one spectrometric-based prediction data object from the at least one trained machine learning computing model; and
perform one or more prediction-based data operations based at least in part on the at least one spectrometric-based prediction data object.

9. The computer program product of claim 8, wherein, when generating the plurality of exhaled breath spectrometric data objects, the computer-readable program code portions comprise the executable portion configured to:
extract at least one of photographic metadata or spectrometric metadata from the exhaled breath digital image data object; and
generate the plurality of exhaled breath spectrometric data objects based at least in part on the at least one of photographic metadata or spectrometric metadata.

10. The computer program product of claim 8, wherein the at least one trained machine learning computing model comprises at least one trained classification-based estimation model.

11. The computer program product of claim 10, wherein, prior to inputting the plurality of exhaled breath spectrometric data objects to the at least one trained machine learning computing model, the computer-readable program code portions comprise the executable portion configured to:
train at least one classification-based estimation model.

12. The computer program product of claim 11, wherein, when training the at least one classification-based estimation model, the computer-readable program code portions comprise the executable portion configured to:
retrieve a plurality of training exhaled breath spectrometric data objects associated with a training spectrometric-based prediction data object;
input the plurality of training exhaled breath spectrometric data objects to the at least one classification-based estimation model;
receive a testing spectrometric-based prediction data object from the at least one classification-based estimation model; and
adjust the at least one classification-based estimation model based at least in part on the testing spectrometric-based prediction data object and the training spectrometric-based prediction data object.

13. The computer program product of claim 8, wherein, when performing the one or more prediction-based data operations based at least in part on the at least one spectrometric-based prediction data object, the computer-readable program code portions comprise the executable portion configured to:
determine whether the at least one spectrometric-based prediction data object satisfies a health condition threshold; and
in response to determining that the at least one spectrometric-based prediction data object satisfies the health condition threshold, transmit a predicted health condition indication to a non-invasive breath analyzer server.

14. The computer program product of claim 8, wherein, when performing the one or more prediction-based data operations based at least in part on the at least one spectrometric-based prediction data object, the computer-readable program code portions comprise the executable portion configured to:
retrieve a previous spectrometric-based prediction data object associated with a previous time point;
retrieve a subsequent spectrometric-based prediction data object associated with a subsequent time point; and
generate a predicted condition progression indication based at least in part on comparing the subsequent spectrometric-based prediction data object with the previous spectrometric-based prediction data object.

15. A computer-implemented method comprising:
receiving an exhaled breath digital image data object from an image generating device;
generating a plurality of exhaled breath spectrometric data objects based at least in part on the exhaled breath digital image data object;
inputting the plurality of exhaled breath spectrometric data objects to at least one trained machine learning computing model;
receiving at least one spectrometric-based prediction data object from the at least one trained machine learning computing model; and
performing one or more prediction-based data operations based at least in part on the at least one spectrometric-based prediction data object.

16. The computer-implemented method of claim 15, wherein, when generating the plurality of exhaled breath spectrometric data objects, the computer-implemented method further comprises:
extracting at least one of photographic metadata or spectrometric metadata from the exhaled breath digital image data object; and
generating the plurality of exhaled breath spectrometric data objects based at least in part on the at least one of photographic metadata or spectrometric metadata.

17. The computer-implemented method of claim 15, wherein the at least one trained machine learning computing model comprises at least one trained classification-based estimation model.

18. The computer-implemented method of claim 17, wherein, prior to inputting the plurality of exhaled breath spectrometric data objects to the at least one trained machine learning computing model, the computer-implemented method further comprises:
training at least one classification-based estimation model.

19. The computer-implemented method of claim 18, wherein, when training the at least one classification-based estimation model, the computer-implemented method further comprises:
retrieving a plurality of training exhaled breath spectrometric data objects associated with a training spectrometric-based prediction data object;
inputting the plurality of training exhaled breath spectrometric data objects to the at least one classification-based estimation model;
receiving a testing spectrometric-based prediction data object from the at least one classification-based estimation model; and
adjusting the at least one classification-based estimation model based at least in part on the testing spectrometric-based prediction data object and the training spectrometric-based prediction data object.

20. The computer-implemented method of claim 15, wherein, when performing the one or more prediction-based data operations based at least in part on the at least one spectrometric-based prediction data object, the computer-implemented method further comprises:
determining whether the at least one spectrometric-based prediction data object satisfies a health condition threshold; and
in response to determining that the at least one spectrometric-based prediction data object satisfies the health condition threshold, transmitting a predicted health condition indication to a non-invasive breath analyzer server.
